(19) United States
(12) Patent Application Publication
(10) Pub. No.: US 2010/0221708 A1
(43) Pub. Date: Sep. 2, 2010

(54) METHOD FOR CHROMOSOME ENUMERATION

(76) Inventors: N. Alice Yamada, San Jose, CA (US); Peter Tsang, San Francisco, CA (US); Robert A. Ach, San Francisco, CA (US); Amir Ben-Dor, Kfar Kava (IL)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/396,308

(22) Filed: Mar. 2, 2009

(51) Int. Cl. C12Q 1/68

(52) U.S. Cl. 435/6

(57) ABSTRACT

A method of sample analysis is provided. The method comprises: a) contacting a genomic sample comprising a plurality of intact chromosomes, i.e., metaphase or interphase chromosomes, with a first set of labeled oligonucleotide probes under in situ hybridization conditions to produce a contacted sample comprising labeled chromosomes, where i. each of the labeled oligonucleotide probes is complementary to a non-repetitive, unique sequence in a region that flanks the centromere of a single chromosome of the plurality of chromosomes; and ii. hybridization of the labeled oligonucleotide probes to the chromosomes produces a distinct labeling pattern for each hybridized chromosome, thereby allowing each of the labeled chromosomes to be distinguished from one another; b) imaging the hybridized chromosomes to provide an image showing the labeling pattern for each labeled chromosome; and c) enumerating a labeled chromosome based on the labeling pattern of said labeled chromosome. A composition and kits for performing the method are also provided.

### BACKGROUND

Chromosome enumeration is a critical application in genomic DNA analysis. Because genomic instability leads to complex patterns of chromosomal aberrations including changes in copy number of whole chromosomes (aneuploidy and/or polypoidy) in certain cells, such as cancer cells, it is important to accurately measure chromosome counts. Also, small pieces of DNA that replicate independently of the regular set of chromosomes, known as marker chromosomes, require enumeration in order to determine the origin of these episome-like small chromosome-like entities.

Chromosome enumeration is also used as a control for fluorescence in situ hybridization (FISH) analysis of amplification and/or deletion of specific loci. FISH allows for the detection of the presence or absence of specific DNA sequences on chromosomes by using fluorescent probes that bind to only those parts of the chromosome with which they show a high degree of complementarity. In these applications, chromosome enumeration is used to differentiate between whole chromosome copy number changes and the localized genomic aberrations. This differentiation allows specific prognosis and/or preferred treatment options for certain genetic aberrations.

Currently, solutions for chromosome enumeration rely largely on probes generated to the α satellite repeat regions within the centromeric domain. Unlike chromosome arms that can recombine or break off, centromeres act as stable targets for chromosome enumeration. Because of their compact nature, there is no recombination at centromeres. There are generally enough differences within low-complexity α satellite repeat regions for there to be specific probes for each chromosome. BAC clones or plasmids that contain such regions can be used as templates to generate FISH probes over these repeat regions.

However, these probes that target α satellite repeat regions cannot accurately differentiate all human chromosomes. In particular, centromeres of chromosomes 13 and 21 or 14 and 22 have such high level of similarity that it is not possible to obtain specific probes. Because centromeric probes target repetitive regions, other probes are also susceptible to cross-hybridization. In order to minimize cross-hybridization, the optimal hybridization conditions may be different between the centromeric probes and non-centromeric probes, complicating experiments that require both probes to be co-hybridized. A large unmet need exists to develop technical methods to enumerate chromosomes accurately while also being able to efficiently detect specific chromosomal abnormalities.

Certain aspects of this disclosure address these needs and describe a method and kits for practicing the same.

### SUMMARY

A method of sample analysis is provided. The method comprises: a) contacting a genomic sample comprising a plurality of intact, i.e., interphase or metaphase, chromosomes with a first set of labeled oligonucleotide probes under in situ hybridization conditions to produce a contacted sample comprising labeled chromosomes, where i. each of the labeled oligonucleotide probes is complementary to a non-repetitive, unique sequence in a region that flanks the centromere of a single chromosome of the plurality of chromosomes; and ii. hybridization of the labeled oligonucleotide probes to the chromosomes produces a distinct labeling pattern for each hybridized chromosome, thereby allowing each of the labeled chromosomes to be distinguished from one another; b) imaging the hybridized chromosomes to provide an image showing the labeling pattern for each labeled chromosome; and c) enumerating a labeled chromosome based on the labeling pattern of said labeled chromosome. A composition and kits for performing the method are also provided.

### BRIEF DESCRIPTION OF THE FIGURES

FIG. 1 schematically illustrates certain features of various chromosomes.

FIG. 2 schematically illustrates certain features of one embodiment of the method described herein.

### DEFINITIONS

The term “sample” as used herein relates to a material or mixture of materials, typically, although not necessarily, in liquid form, containing one or more analytes of interest.

The term “genomic sample” as used herein relates to a material or mixture of materials, containing genetic material from an organism. The term “genomic DNA” as used herein refers to deoxyribonucleic acids that are obtained from an organism. The terms “genomic sample” and “genomic DNA” encompass genetic material that may have undergone amplification, purification, or fragmentation. The term “test genome,” as used herein refers to genomic DNA that is of interest in a study.

The term “nucleotide” is intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the term “nucleotide” includes those moieties that contain hapten or fluorescent labels and may contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, are functionalized as ethers, amines, or the likes.

The term “nucleic acid” and “polynucleotide” are used interchangeably herein to describe a polymer of any length, e.g., greater than about 2 bases, greater than about 10 bases, greater than about 100 bases, greater than about 500 bases, greater than 1000 bases, up to about 10,000 or more bases composed of nucleotides, e.g., deoxyribonucleotides or ribonucleotides, and may be produced enzymatically or synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions. Naturally-occurring nucleotides include guanine, cytosine, adenine and thymine (G, C, A and T, respectively).

The term “oligonucleotide” as used herein denotes a single stranded multimer of nucleotide of from about 2 to 200 or more, up to about 500 nucleotides or more. Oligonucleotides may be synthetic or may be made enzymatically, and, in some embodiments, are less than 10 to 50 nucleotides in length. Oligonucleotides may contain ribonucleotide monomers (i.e., may be oligoribonucleotides) or deoxyribonucleotide monomers. Oligonucleotides may be 10 to 20, 11 to 30, 31 to 40, 41 to 50, 51-60, 61 to 70, 71 to 80, 80 to 100, 100 to 150 or 150 to 200 nucleotides in length, for example.

The term “sequence-specific oligonucleotide” as used herein refers to an oligonucleotide that only binds to a single site in a haploid genome. In certain embodiments, a “sequence-specific” oligonucleotide may hybridize to a complementary nucleotide sequence that is unique in a sample under study.

The term “complementary” as used herein refers to a nucleotide sequence that base-pairs by non-covalent bonds to a target nucleic acid of interest. In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is fully complementary to a target of interest such that every nucleotide in the sequence is complementary to every nucleotide in the target nucleic acid in the corresponding positions. In certain cases, a nucleotide sequence may be partially complementary to a target, in which not all nucleotide is complementary to every nucleotide in the target nucleic acid in all the corresponding positions.

The term “probe”, as used herein, refers to a nucleic acid that is complementary to a nucleotide sequence of interest. In certain cases, detection of a target analyte requires hybridization of a probe to a target. In certain embodiments, a probe may be immobilized on a surface of a substrate, where the substrate can have a variety of configurations, e.g., a sheet, bead, or other structure. In certain embodiments, a probe may be present on a surface of a planar support, e.g., in the form of an array.

An “array” includes any two-dimensional or substantially two-dimensional (as well as a three-dimensional) arrangement of addressable regions, e.g., addressable regions, e.g., spatially addressable regions or optically addressable regions, bearing nucleic acids, particularly oligonucleotides or synthetic mimetics thereof, and the like. Where the arrays are arrays of nucleic acids, the nucleic acids may be adsorbed, physisorbed, chemisorbed, or covalently attached to the arrays at any point or points along the nucleic acid chain.

Any given substrate may carry one, two, four or more arrays disposed on a surface of the substrate. Depending upon the use, any or all of the arrays may be the same or different from one another and each may contain multiple spots or features. An array may contain one or more, including more than two, more than ten, more than one hundred, more than one thousand, more ten thousand features, more than one hundred thousand features, or even more than million features, in an area of less than 20 cm² or even less than 10 cm², e.g., less than about 5 cm², including less than about 1 cm², less than about 1 mm², e.g., 100 μm², or even smaller. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different compositions (for example, when any repeats of each feature composition are excluded the remaining features may account for at least 5%, 10%, 20%, 50%, 95%, 99% or 100% of the total number of features). Inter-feature areas will typically (but not essentially) be present which do not carry any nucleic acids (or other biopolymer or chemical moiety of a type of which the features are composed). Such inter-feature areas typically will be present where the arrays are formed by processes involving drop deposition of reagents but may not be present when, for example, photolithographic array fabrication processes are used. It will be appreciated though, that the inter-feature areas, when present, could be of various sizes and configurations.

Each array may cover an area of less than 200 cm², or even less than 50 cm², 5 cm², 1 cm², 0.5 cm², or 0.1 cm². In certain embodiments, the substrate carrying the one or more arrays will be shaped generally as a rectangular solid (although other shapes are possible), having a length of more than 4 mm and less than 150 mm, usually more than 4 mm and less than 80 mm, more usually less than 20 mm; a width of more than 4 mm and less than 150 mm, usually less than 80 mm and more usually less than 20 mm; and a thickness of more than 0.01 mm and less than 5.0 mm, usually more than 0.1 mm and less than 2 mm and more usually more than 0.2 mm and less than 1.5 mm, such as more than about 0.8 mm and less than about 1.2 mm.

Arrays can be fabricated using drop deposition from pulse-jets of either precursor units (such as nucleotide or amino acid monomers) in the case of in situ fabrication, or the previously obtained nucleic acid. Such methods are described in detail in, for example, the previously cited references including U.S. Pat. No. 6,242,266, U.S. Pat. No. 6,232,072, U.S. Pat. No. 6,180,351, U.S. Pat. No 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods can be used for fabrication, as previously described herein. Also, instead of drop deposition methods, photolithographic array fabrication methods may be used. Inter-feature areas need not be present particularly when the arrays are made by photolithographic methods as described in those patents.

Arrays may also be made by distributing pre-synthesized nucleic acids linked to beads, also termed microspheres, onto a solid support. In certain embodiments, unique optical signatures are incorporated into the beads, e.g. fluorescent dyes, which could be used to identify the chemical functionality on any particular bead. Since the beads are first coded with an optical signature, the array may be decoded later, such that correlation of the location of an individual site on the array with the probe at that particular site may be made after the array has been made. Such methods are described in detail in, for example, U.S. Pat. Nos. 6,355,431, 7,033,754, and 7,060,431.

An array is “addressable” when it has multiple regions of different moieties (e.g., different oligonucleotide sequences) such that a region (i.e., a “feature” or “spot” of the array) at a particular predetermined location (i.e., an “address”) on the array contains a particular sequence. Array features are typically, but need not be, separated by intervening spaces. An array is also “addressable” if the features of the array each have an optically detectable signature that identifies the moiety present at that feature.

The terms “determining”, “measuring”, “evaluating”, “assessing”, “analyzing”, and “assaying” are used interchangeably herein to refer to any form of measurement, and include determining if an element is present or not. These terms include both quantitative and/or qualitative determinations. Assessing may be relative or absolute. “Assessing the presence of” includes determining the amount of something present, as well as determining whether it is present or absent.

The term “using” has its conventional meaning, and, as such, means employing, e.g., putting into service, a method or composition to attain an end. For example, if a program is used to create a file, a program is executed to make a file, the file usually being the output of the program. In another example, if a computer file is used, it is usually accessed, read, and the information stored in the file employed to attain an end. Similarly if a unique identifier, e.g., a barcode is used, the unique identifier is usually read to identify, for example, an object or file associated with the unique identifier.

The term “intact chromosome”, as used herein, refers to an organized structure of DNA and proteins found in mammalian cells during the metaphase phase (i.e., a “metaphase chromosome”) or interphase phase (i.e., an interphase chromosome) of the cell cycle. Each intact chromosome has two arms, in which one arm is the p arm and the other is the q arm. Each intact chromosome contains two sister chromatids that are joined together by a centromere.

The term “chromosomal rearrangement”, as used herein, refers to an event where one or more parts of a chromosome are rearranged within a single chromosome or between chromosomes relative to a reference chromosome. In certain cases, a chromosomal rearrangement may reflect an abnormality in chromosome structure. A chromosomal rearrangement may be an inversion, a deletion, an insertion or a translocation, for example.

The term “contacting” means to bring or put together. As such, a first item is contacted with a second item when the two items are brought or put together, e.g., by touching them to each other or combining them in the same solution. Thus, a “contacted sample” is a test chromosome onto which oligonucleotide probes have been hybridized.

The term “hybridization” refers to the specific binding of a nucleic acid to a complementary nucleic acid via Watson-Crick base pairing. Accordingly, the term “in situ hybridization” refers to specific binding of a nucleic acid to a metaphase or interphase chromosome.

The terms “hybridizing” and “binding”, with respect to nucleic acids, are used interchangeably.

The terms “plurality”, “set” or “population” are used interchangeably to mean at least 2, at least 10, at least 100, at least 500, at least 1000, at least 10,000, at least 100,000, at least 1000,000, at least 10,000,000 or more.

The term “in situ hybridization conditions” as used herein refers to conditions that allow hybridization of a nucleic acid to a complementary nucleic acid in an intact chromosome. Suitable in situ hybridization conditions may include both hybridization conditions and optional wash conditions, which include temperature, concentration of denaturing reagents, salts, incubation time, etc. Such conditions are known in the art.

As used herein, the term “repetitive” refers to a nucleotide sequence that contains a repeated unit composed of at least two different nucleotides, where the unit is repeated in tandem for at least 20 times without any intervening nucleotides. Each unit of a repeat may be of a length of at least 2-200 nucleotides, or more (e.g. a length in the range of 5-10 nucleotides, 10-50 nucleotides, or 100-200 nucleotides, or more). In a repetitive sequence, the repeating unit repeats in tandem for at least 20 times (e.g. up to 50 times, up to 1,000, up to 10,000 times, up to 100,000 times or more) in a contiguous sequence. (X-satellite DNA, LINES, and SINES are types of repetitive sequences.

As used herein, the term “non-repetitive” refers to a sequence that is not repetitive, as discussed above.

As used herein, the term “unique” refers to a sequence in a region of a chromosome that exists no more than once in a plurality of unduplicated chromosomes in a genomic sample. In duplicated chromosomes, a unique probe may bind to two sister chromatids of the same chromosome at the same locations.

As used herein, the term “a region that flanks the centromere” or “a centromere-flanking region” refers to a chromosomal region that is up to about 5 Mb (e.g. up to 2 Mb, up to 1 Mb, up to 100 kb, up to 10 kb, up to 2 kb or less) upstream or downstream to a centromere of a chromosome. The length of the region may be 1 kb to 100 kb (e.g. 1 kb to 50 kb, 5 kb to 100 kb, or 10 kb to 50 kb).

As used herein, the term “distal to the centromere” refers to a region more than about 1 Mb (e.g. more than 2 Mb, more than 5 Mb, or more) from the centromere.

A “labeling pattern” refers to the pattern of labels that is generated in an image when labeled probes hybridized to an intact chromosome are visualized. The labeling pattern in an image is derived from wavelength and spatial components collected as data by a detecting apparatus (e.g. microscope). A “distinct labeling pattern” or “distinctly labeled”, as used herein, refers to a labeling pattern of a labeled chromosome that is different from labeling patterns of other chromosomes of a different type.

The term “overlapping”, as used herein in reference to probes, refers to sequence of one probe that is partly or entirely shared with the sequence of another probe. For example, in a first and a second probes that are overlapping, the sequence of the 3′ region of the first probe may be the same as that of the 5′ region of the second probe.

The term “enumeration” or “enumerate”, as used herein, refers to the identification of a chromosome from an organism according to the nomenclature established for the organism in the field of chromosomal research. For example, human chromosomes are enumerated 1 to 22, plus the sex chromosomes, in accordance with the numbering and nomenclature system established by the International System for Human Cytogenetic Nomenclature (ISCN).

### DESCRIPTION OF EXEMPLARY EMBODIMENTS

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Method of Sample Analysis

A method for analyzing a genome is provided. In certain embodiments, the method includes: a) contacting a genomic sample comprising a plurality of intact, e.g., metaphase or interphase, chromosomes with a first set of labeled oligonucleotide probes under in situ hybridization conditions to produce a contacted sample comprising labeled chromosomes, where i. each of the labeled oligonucleotide probes is complementary to a non-repetitive, unique sequence in a region that flanks the centromere of a single chromosome of the plurality of chromosomes; and ii. hybridization of the labeled oligonucleotide probes to the chromosomes produces a distinct labeling pattern for each hybridized chromosome, thereby allowing each of the labeled chromosomes to be distinguished from one another; b) imaging the hybridized chromosomes to provide an image showing the labeling pattern for each labeled chromosome; and c) enumerating a labeled chromosome based on the labeling pattern of said labeled chromosome.

Certain features of the subject method are illustrated in FIG. 2 and are described in greater detail below. In certain embodiments, the method involves contacting 12 a genomic sample with a set of sequence-specific oligonucleotide probes 18 under in situ hybridization conditions. The probes are linked to detectable labels and hybridize to specific sequences in regions flanking the centromeres of intact chromosomes 16 in the genomic sample. The plurality of intact chromosomes becomes labeled 20 as a result of the contacting step 12 in a sequence-specific manner. The labels then may be detected, e.g., using a microscope, to provide an image of the labeled chromosomes. Each chromosome imaged may be assigned a chromosome number based on its own distinct labeling pattern. A similar method may be applied to analyze a sample containing interphase chromosomes.

In order to generate a distinct labeling pattern so as to allow each chromosome to be distinguished from one another, the labeled oligonucleotide probes may have following features. Each labeled oligonucleotide probe is designed to be complementary to no more than one chromosomal region such that each probe is unique to one location in one unduplicated chromosome out of a plurality of chromosomes. Each probe is also linked to a specific type of label out of a variety of detectable labels (e.g. different fluorescent labels). As such, a specific combination of different probes may be designed for each chromosome in order to generate a unique labeling pattern for that chromosome. The labeling pattern for each specific chromosome can be predicted by the sequence of the probes and the sequence of the chromosomes. Enumeration of the chromosome then involves matching the labeling pattern of an actual hybridized chromosome with the predicted labeling pattern. For example, if an unknown chromosome exhibits a labeling pattern predicted for chromosome 3, the unknown chromosome would be enumerated as chromosome 3.

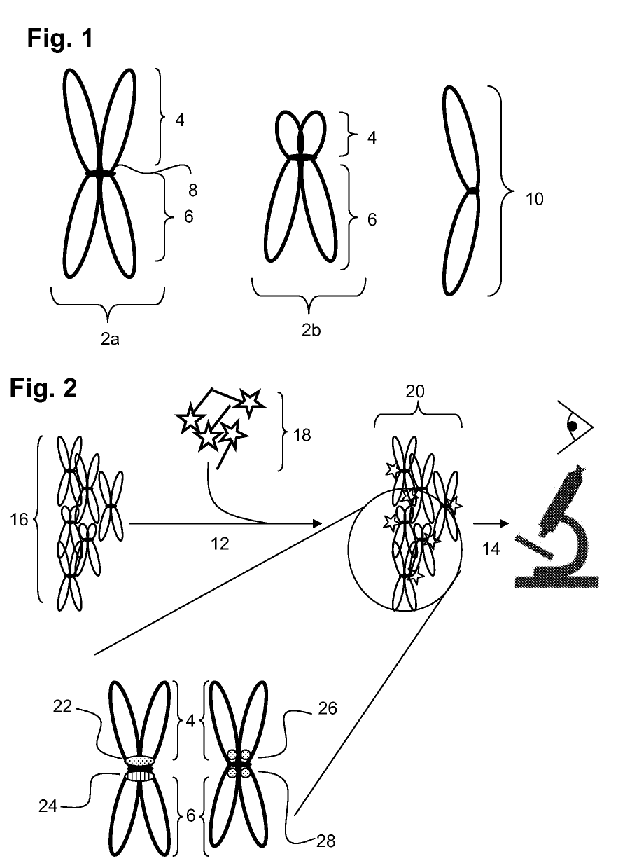

Certain features of the oligonucleotide probes employed in the subject method are described in greater detail below. One feature of the probe is that it is complementary to sequences in regions flanking the centromere as opposed to the centromeres themselves. The centromere, as shown as element 8 in FIG. 1, is where sister chromatids, one of which shown separately as element 10, are joined together. The centromere comprises a DNA structure with proteins assembled on highly repetitive and AT-rich sequences. Human centromere, for example, spans about 0.3 to about 5 Mbp of DNA that contains extensive (about 1,500 to more than 30,000 copies), tandemly repeated arrays of a 171 bp sequence element termed α satellite. Most often than not, these high repetitive and AT-rich characteristics are shared among centromeres of all chromosomes of a plurality in a genomic sample. Consequently, probes designed to be complementary to a centromere of a specific chromosome may easily cross-hybridize among a plurality of chromosomes. Since probes employed in the subject method are complementary to regions flanking the centromere and not to regions of the centromere, the probe sequences are not dependent on hybridizing to centromeres of the chromosomes. As such, the labeled probes employed in the subject method are not likely to cross-hybridize between chromosomes or participate in non-specific hybridization common to highly repetitive and AT-rich sequences.

In certain cases, the specificity of oligonucleotide probes may be increased by selecting complementary sequences of varied nucleotide compositions. Not only can the probe sequence avoid centromeric sequences that are AT-rich and highly repetitive, specific sequences of varying percentages of nucleotide composition or of varying lengths may also be selected to increase the hybridization efficiency of the oligonucleotide probes to the chromosomes. Programs and tools that can predict melting temperature and specificity of various sequences are prevalent in the art and may be used in designing specific oligonucleotide probes.

One other feature of the oligonucleotide probes employed in the subject method is that the probes are complementary to sequences in regions that flanks the centromere such that they are still close to the centromere in base pair distance. Since the complementary sequences of the probes are in close proximity to the centromere, the sequences are less likely to recombine and relocate than genomic loci distal to the centromere as the chromosomes undergo replication from generation to generation. The assignment of a specific probe sequence to a chromosome number may remain unchanged despite the growth phase, the passage number, or the source of genomic samples.

In certain embodiments, regions that flank the centromere to which the probes are complementary include chromosomal regions of no more than about 5 Mb, e.g. 2 Mb, 1 Mb, 100 kb, 10 kb, 2 kb or less, upstream or downstream to a centromere of a chromosome. For example, the probes may be complementary to sequences within about 2 Mb upstream or downstream to a centromere of a chromosome.

Within these regions that flank the centromere, the specific location of where the probes bind relative to the centromere may further aid in generating different labeling patterns. As shown in FIG. 1, centromere 8 is where the two sister chromatids are joined and it also divides each sister chromatid into either p arm 4 or q arm 6. Both arms of the two sister chromatids are physically very close to each other in the centromeric region. The more distanced the regions are from the centromere, the more separate spatially the arms are from each other. Consequently, depending on the location of where the labeled probes bind along the p or q arms of the two sister chromatids, the signals from the two sister chromatids of an intact metaphase chromosome may appear spatially distinct or spatially indistinct under a microscope used for in situ hybridization experiments. If the probes are complementary to sequences sufficiently proximal to the centromere, the signals from the sister chromatids of the labeled chromosome may appear spatially indistinct. In other embodiments, if the labeled oligonucleotide probes bind to a region that is sufficiently distal to the centromere, the signals from the sister chromatids chromosome may appear spatially distinct. To demonstrate how spatially indistinct or distinct signals may appear on labeled intact metaphase chromosomes, two exemplary chromosomes are illustrated in FIG. 2 as a hypothetical zoomed image from the plurality of labeled chromosomes 20. For example, as shown in FIG. 2, one chromosome has spatially indistinct signals, 22 and 24, from the two sister chromatids on both the p arms and the q arms, while the other chromosome has spatially distinct signals 26 and 28 on the p arm and the q arms, respectively.

In combination with the type of labels linked to the oligonucleotide probe and other features of the probe, the ability to generate spatially distinct or indistinct signals on either the p arm or the q arm allows unique labeling patterns to be designed for each of a plurality of chromosomes. The subject method involves hybridizing the labeled oligonucleotide probes described above to a sample containing intact chromosomes and analyzing their distinct labeling patterns. An intact metaphase or interphase chromosome may be prepared for the subject method in accordance with the many established protocols known to one of ordinary skill in the art (e.g. Wiegant J and Raap A K. Curr Protoc Cytom. 2001 May, Chapter 8: Unit 8.2; Bayani J and Squire J A. Curr Protoc Cell Biol. 2004 September, Chapter 22: Unit 22.4). The chromosomes are then attached to a substrate, e.g., glass, and contacted with labeled oligonucleotide probes under in situ hybridizing conditions. “In situ hybridizing conditions” are conditions that facilitate annealing between a nucleic aid and the complementary nucleic acid in intact chromosomes. Hybridization conditions vary, depending on the concentrations, base compositions, complexities, and lengths of the probes, as well as salt concentrations, temperatures, and length of incubation. For example, in situ hybridizations may be performed in hybridization buffer containing 1-2×SSC and 50% formamide. In certain cases, blocking DNA such as cot-1 DNA is not used to suppress non-specific hybridization in the in situ hybridization method. In general, hybridization conditions include temperatures of about 25° C. to about 55° C., and incubation times of about 0.5 hours to about 96 hours. An appropriate counterstain that enhances the detection of the labeled oligonucleotide probe may also be chosen to stain the chromosomes (e.g. DAPI, Hoechst 33258, or propidium iodide). In situ hybridization assays and methods for sample preparation are well known to those of skill in the art and need not be described in detail here.

In certain embodiments, only one arm of the centromere set may be hybridized to identify enumerate the state of interphase chromosome content. Because interphase cells can be in various stages of DNA synthesis, it is possible for interphase cells to be in 2N or 4N genomic content state. It is useful to determine whether cells are in 2N or 4N state in interphase cells when conducting FISH for other genomic locations of interest in combination with the use of these centromere probes. Both the flanking set or the p- or q-arm probe used singularly may be employed in this application to determine the state of replication. When the flanking set is used on the interphase cells, the read out may be as simple as determining whether there are four spots or eight spots.

The contacted sample can be read or imaged using a variety of different techniques, for example, by microscopy, flow cytometry, fluorimetry, etc. Microscopy, for example light microscopy, fluorescent microscopy or confocal microscopy, is an established analytical tool for detecting light signals from a sample. In embodiments in which oligonucleotides are labeled with a fluorescent moiety, reading of the contacted sample may be carried out by fluorescence microscopy. Fluorescent microscopy or confocal microscopy used in conjunction with fluorescent microscopy may also be used. The microscopy device can include a temperature controller to maintain the sample at a specific temperature while it is being scanned. A multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data and images collected during the assay into another format for presentation. In general, known robotic systems and components can be used.

Such methods are generally known in the art and may be readily adapted for use herein. For example, the following references discuss chromosome hybridization: Ried et al., Chromosome painting: a useful art Human Molecular Genetics, Vol 7, 1619-1626; Speicher et al: Karyotyping human chromosomes by combinatorial multi-fluor FISH, Nature Genetics, 12, 368-376, 1996; Schrock et al: Multicolor Spectral Karyotyping of Human Chromosomes. Science, 494-497, 1996; Griffin et al Molecular cytogenetic characterization of pancreas cancer cell lines reveals high complexity chromosomal alterations. Cytogenet Genome Res. 2007; 118(2-4):148-56; Peschka et al, Analysis of a de novo complex chromosome rearrangement involving chromosomes 4, 11, 12 and 13 and eight breakpoints by conventional cytogenetic, fluorescence in situ hybridization and spectral karyotyping. Prenat Diagn. 1999 December; 19(12):1143-9; Hilgenfeld et al, Analysis of B-cell neoplasias by spectral karyotyping (SKY). Curr Top Microbiol Immunol. 1999; 246: 169-74. Ried et al, Genomic changes defining the genesis, progression, and malignancy potential in solid human tumors: a phenotype/genotype correlation. Genes Chromosomes Cancer. 1999 July; 25(3):195-204; and Agarwal et al, Comparative genomic hybridization analysis of human parathyroid tumors. Cancer Genet Cytogenet. Oct. 1, 1998; 106(1):30-6.

Employing the labeled oligonucleotide probes described previously in accordance with the subject method allows the enumeration of chromosomes based on the image of the labeling pattern produced by hybridizing the probes to intact metaphase or interphase chromosomes. In certain cases, enumerating comprises determining the emission spectrum of the labels linked to the hybridized probes. In one embodiment, the emission spectrum may contain a maximum at 521 nm like fluorescein isothiocyanate or a maximum at 617 nm like Alexa Fluor® 594. In another embodiment, the emission spectrum may contain a maximum at −570 nm like Cy3 or at >650 nm like Cy5. In certain cases, an image of a region flanking the centromere of a labeled chromosome may be of one color, two different colors, or more than two different colors. Additional fluorophores or detectable labels are also contemplated herein and will be discussed later. The apparatus used for detecting the labels of probes hybridized to the chromosomes may include, but not limited to microscope, array scanner, flow cytometry, etc. Different filter sets or lasers may be required depending on the fluorescent moiety used. As an exemplary example, if only green and red fluorescent moieties are used in the labeled oligonucleotide probes, only two types of filter sets may be required to detect the hybridized labeled oligonucleotide probes and possibly another filter set may be used to detect the counterstain of chromosomes under a microscope.

In certain embodiments, enumerating comprises determining whether the labeling pattern of a chromosome comprises a spatially distinct or spatially indistinct signal from the two sister chromatids. In certain cases, the determining may depend on the resolution of the apparatus used for detecting the labels, the image taken of the hybridized chromosomes, and/or the evaluation of the image by a human operator or a computer program. In one embodiment, the resolution of the apparatus (e.g. fluorescence microscope) may at least be about 500 μm (e.g. 100 μm, 100 μm, 50 μm, 10 μm, 1 μm or about 10 nanometers).

In other cases, enumerating comprises determining the morphology of the chromosomes. The chromosome morphology analyzed may include lengths of the p and q arms relative to each other. In such embodiments, the lengths of p and q arms may vary in length depending on the chromosomes such that their lengths may be informative of the chromosome identity. For example, humans have both metacentric chromosomes and acrocentric chromosomes. Metacentric chromosomes have both arms roughly equal in length, shown as chromosome 2a in FIG. 1. In acrocentric chromosomes, the p arm is much shorter than the q arm but still present, exemplified by chromosome 2b in FIG. 1. There are five acrocentric chromosomes in the human genome: 13, 14, 15, 21 and 22. Hence, in certain embodiments for a human genomic sample, an acrocentric chromosome may be enumerated as 13, 14, 15, 21, or 22. In many embodiments, probes hybridized to regions flanking the centromeres on the p arms of acrocentric chromosomes are detected as spatially indistinct signals. This is because the p arms are too short to select a sequence in the region flanking the centromere that is sufficiently distal from the centromere in order for the signals from the two sister chromatids to appear distinct. As such, the signals from the two sister chromatids on the p arms of an acrocentric chromosome are spatially indistinct.

The subject method described herein involves having a distinct labeling pattern for each labeled chromosome of a plurality. To generate distinct labeling patterns, several factors may be varied, such as the location of the complementary sequence of the probes to produce spatially distinct or indistinct signals on the sister chromatids and the types of detectable labels, as discussed above. Details involved in generating distinct labeling patterns from these variables will be discussed in an embodiment illustrated below.

In an embodiment where metaphase or interphase chromosomes from a human genomic sample are to be enumerated according to the subject method, distinct labeling patterns may be generated for all 24 distinct human chromosomes (1-22 in addition to the two sex chromosomes) using two different types of detectable label. For example, red and green fluorescent moieties may be used as the two types of detectable labels for the oligonucleotide probes. For either the p or q arms, the arms may be hybridized to red probes, green probes or probes labeled with both red and green fluorescent moieties. Hence, the arms may be labeled with one of the following three “colors”: red, green, and red/green. As noted above, the hybridized p arms of acrocentric chromosomes can only be labeled as spatially indistinct signals due to their short lengths. However, the q arms of the acrocentric chromosomes may be labeled to have either spatially indistinct signals or spatially distinct signals, such as 24 or 28 in FIG. 2. These two patterns of spatially indistinct and spatially distinct signals combined with three different “colors” allow the q arms to have 6 distinct patterns of labeling. Together with the 3 different “color” labeling of the p arms, there can be 18 different labeling patterns for acrocentric chromosomes.

As for metacentric chromosomes that do not have a shorter p arm relative to the q arm, both the p and the q arms may have either spatially indistinct or distinct signals (22 or 26 for the p arms and 24 or 28 for the q arms, as shown in FIG. 2). Together with 3 different “color” labeling, there can be 6 patterns of labeling for p arm and 6 patterns for the q arm. Since one pattern needs to be selected for the p arms and another for the q arms to generate a distinct labeling pattern for one whole chromosome, a two-pattern combination needs to be selected for one chromosome labeling pattern. The number of possible distinct combinations in which p and q arms are labeled differently from each other is

$$( \begin{matrix}6 \\ 2\end{matrix} ) = 6 * 5 / 2 = 15$$

In this embodiment, there are 15 distinct combinations when the p and q arms are labeled differently. In addition to these 15 distinct combinations, there are additional 6 labeling patterns if the p arms and q arms are labeled exactly the same way. Combining the 15 labeling patterns with the 6 labeling patterns, there are 21 distinct labeling patterns for non-acrocentric chromosomes.

Based on the embodiment above, there are 18 distinct labeling patterns for acrocentric chromosomes and 21 distinct labeling patterns for non-acrocentric chromosomes. These numbers of distinct labeling patterns are more than enough to label distinctly each of the 24 distinct human chromosomes, in which 5 are acrocentric.

In a case where more than two different types of detectable labels are used in the subject method, more “color” options would be available than the embodiment described above. Hence, even more distinct labeling patterns may be generated than the ones generated in the embodiment above. In a situation where two different types of detectable label do not provide enough unique labeling patterns to adequately label all chromosomes in a plurality, more than two different types of detectable label may be used. For example, additional labels can be used to give more colors, e.g., 3 labels gives 7 distinguishable signals (the three individual colors, three combinations of two colors, and one combination of all three colors), four labels gives 15 distinguishable signals, and so on. Moreover, depending on the morphology of the chromosomes in the genomic sample, different number of distinct labeling patterns for acrocentric or non-acrocentric chromosomes may be required and the number of “colors” may be adjusted accordingly.

In certain embodiments, the method allows contacting a genomic sample comprising a plurality of metaphase or interphase chromosomes to a set of labeled oligonucleotide probes in which all probes of the set are contacted to the chromosomes in the same time. Based on the specificity of the probes and the melting temperatures of the resultant duplexes, all of the probes in the set may be designed to hybridize to their targets under the same hybridization conditions. For example, all the duplexes resulting from hybridizing the probes to their complementary sequences may be Tₘ-matched.

In other embodiments, the method allows contacting chromosomes to a second set of probes that are complementary to genomic loci distal to the centromere in addition to the first set of labeled oligonucleotide probes discussed previously. The first set of probes is employed for the purpose for enumerating chromosomes while the second set may be used to detect the presence of a genetic element. The second set of probes designed to hybridize to genomic loci distal to the centromere are complementary to regions more than at least 1 Mb (e.g. at least about 2 Mb, about 5 Mb, or more) from the centromeres. In certain embodiments, the genomic loci distal to the centromere contain allele specific markers, predicted or known coding regions, or other genetic elements of interest. In other embodiments, recombination events can be detected as the genomic loci are located sufficiently far away in base pair distance from the centromeres. In certain cases, the hybridization conditions are the same for both the first set and the second set of probes, and therefore, the contacting to both sets may be done sequentially or in the same time. In certain embodiments, all the duplexes resulting from hybridizing both the first set and the second set of probes to their complementary sequences may be Tₘ-matched. As such, the subject method may facilitate multiplexing a plurality of genomic samples such that chromosomes may be enumerated in accordance with the subject method and analyzed for other genomic loci all in the same time.

In certain embodiments, the labeling pattern of a hybridized chromosome may be compared with that of a reference chromosome. The reference chromosome may be a chromosome derived from a supposedly healthy or wild-type organism and hybridized to the labeled probes. In another embodiment, the reference chromosome may be a hypothetical chromosome with a predicted labeling pattern for a supposedly healthy or wild-type organism. The labeling pattern of the reference chromosome may be determined before, after or at the same time as the labeling pattern of the test chromosome. The matching of labeling patterns between the test chromosome and the reference chromosome may be performed by using computer-based analysis software known in the art.

In other embodiments, a first set and a second set of probes may be hybridized to both a test chromosome and a reference chromosome. Hybridization of the first set of probes to the chromosomes generates labeling patterns that allow enumeration of chromosomes in accordance with the method described above. Hybridization of the second set of probes to the chromosomes generates signals that indicate the presence or absence of a particular genetic element distal to the centromeres. Knowing the chromosome numbers may aid in selecting images of the chromosome under study and of the corresponding reference chromosome for analysis. Comparing the images of the labeled test chromosome and of the labeled reference chromosome allows the determination of any differences in the binding of the second set of probes between the two chromosomes. Determination may be done manually (e.g., by viewing the data and comparing the labeling signals by hand), automatically (e.g., by employing data analysis software configured specifically to match a labeling pattern), or a combination thereof. In certain embodiments, a difference in the labeling signals between a test chromosome and a reference chromosome may indicate a chromosomal or genetic abnormality.

In certain embodiments, the subject method includes a step of transmitting data from at least one of the detecting and enumerating steps, as described above, to a remote location. By “remote location” is meant a location other than the location at which the array is present and hybridization occur. For example, a remote location could be another location (e.g., office, lab, etc.) in the same city, another location in a different city, another location in a different state, another location in a different country, etc. As such, when one item is indicated as being “remote” from another, what is meant is that the two items are at least in different buildings, and may be at least one mile, ten miles, or at least one hundred miles apart. “Communicating” information means transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. The data may be transmitted to the remote location for further evaluation and/or use. Any convenient telecommunications means may be employed for transmitting the data, e.g., facsimile, modem, internet, etc.

### Composition

As will be described in greater detail below, the oligonucleotide composition described herein may be employed to hybridize to an intact metaphase or interphase chromosome to generate a distinct labeling pattern, thereby allowing each labeled chromosome to be distinguished from one another in a plurality. Each of the labeled oligonucleotide probes in a set is complementary to a non-repetitive, unique sequence in a region that flanks the centromere of a single chromosome of a plurality of chromosomes; and hybridization of the labeled oligonucleotide probes to the chromosomes produces a distinct labeling pattern for each of the chromosomes. The labeling pattern of the oligonucleotides on the chromosome can then be analyzed.

Each set of oligonucleotide probes of the subject composition comprises a pre-determined number of at least 10, at least 100, at least 1000, e.g. at least 5,000, at least 10,000 or at least 50,000, up to 100,000 or more different oligonucleotide probes. In certain embodiments, each oligonucleotide probe may be in the range of 50 to 200 nucleotides in length, or more. In certain embodiments, about 10 to 20, about 20 to 40, about 40 to 80, or more oligonucleotide probes are directed to each region that flanks the centromere of a chromosome.

In certain cases, a certain part of a probe may overlap with parts of other probes, allowing certain probes to overlap when hybridized to a contiguous sequence in a region flanking the centromere of a chromosome. More than 1, 2, 3 or more probes may participate in overlapping a particular sequence. In other cases, the probes may be uniquely tiled (e.g. end-to-end tiling). In certain embodiments, in a group of probes that hybridize to a region flanking the centromere, more than about 5, 10, 50, 80 or more probes would participate in overlapping. The extent of an overlap may be anywhere between about 1 to 10 nucleotides, about 20 to 50 nucleotides, or about 50 to 100 nucleotides. In certain embodiments, for a group of oligonucleotide probes hybridized to a region flanking the centromere of a chromosome, each of the overlapping regions may have an average length of about 50 nucleotides. In other cases, there may be one or more intervening sequences between hybridized probes where no probes are hybridized in that region flanking the centromere. In other cases, there are no intervening sequences between hybridized probes such that the hybridized sequence in a region flanking the centromere is one contiguous sequence.

As discussed above, the oligonucleotide probes comprise sequence complementary to a known, non-repetitive, unique sequence in a region that flanks the centromere of a chromosome. The region may be a chromosomal region of no more than about 5 Mb (e.g. up to 2 Mb, up to 1 Mb, up to 100 kb, up to 10 kb, up to 2 kb or less) upstream or downstream to a centromere of a chromosome. The length of the region may be about up to 100 kb (e.g. up to 50 kb, up to 5 kb, up to 2 kb, or up to 1 kb). There are two regions that flank the centromere of each unduplicated chromosome, one for the p arm and one for the q arm. In a duplicated chromosome, each of the two sister chromatids of the same chromosome would have a region flanking the centromere on the p arm and another on the q arm. In certain embodiments, the region flanking the centromere is greatly suppressed from recombination relative to regions distal to the centromere. It is within the ability of one of ordinary skill in the art to determine a region that flanks the centromere of the chromosome given the availability of not only the physical genomic maps but also of recombination and linkage maps (Kong et al. (2002) Nat. Genetics 31:241-247). For example, the region that flanks the centromere may be 2 Mb upstream or downstream to a centromere of a chromosome.

Within a region that flanks the centromere, one or more non-repetitive sequences are selected as complementary sequences of the probes if the sequence is at least contiguous for at least 1 kb, or 2 kb, or 5 kb or more in length for non-repetitive fragments. Each non-repetitive fragment is composed of at least 100 (e.g. up to at least 150, up to at least 200, up to at least 250, up to at least 300, or more) base pairs in which there are no units composed of at least two different nucleotides that repeat for at least 20 times (e.g. 50 times, 100,000 times, 1 million times or more).

In addition to being complementary to non-repetitive sequences, each of the subject oligonucleotide probes is also unique to a location in an unduplicated chromosome. A probe that is unique to a location to an unduplicated chromosome is neither complementary to other locations within the same unduplicated chromosome nor to locations in other chromosomes. In a duplicated chromosome, a unique probe may bind to two sister chromatids of the same chromosome at the same locations.

In certain embodiments, all the probes of a set are designed to hybridize to the complementary sequences in a region that flanks the centromere under the same hybridization conditions. As noted previously, all the duplexes resulting from hybridizing the probes to their complementary sequences may be Tₘ-matched.

Since the genome sequences of many organisms, including many bacteria, fungi, plants and animals, e.g., mammals such as human, primates, and rodents such as mouse and rat, are known and some are publicly available (e.g., in NCBI's Genbank database), the design of the above-described oligonucleotides and identification of centromeres for the chromosomes under study are all within the skill of one of ordinary skill in the art. Centromeric sequences of many organisms have been extensively studied. Programs and tools are widely available in which many parameters may be used to help identify repetitive sequences in centromeres. Parameters may include length of the sequence, sequence of repeat units, and the number of repeats per unit. Such methods are generally known in the art and may be readily adapted for use herein. For example, the following references discuss analyzing centromeric repeat sequences: Laurent et al. (2003) Human Mole. Genetics 12:2229-2239, Henikoff et al. (2001) Science 293:1098-1102, Willard et al. (1987) Trends in Genetics 3:192-198, Alkan et al. (2007) PloS Computational Biology 3:1807-1818.

In certain embodiments, oligonucleotide probes may be designed using methods set forth in US20040101846, U.S. Pat. No. 6,251,588, US20060115822, US20070100563, US20080027655, US20050282174, U.S. patent application Ser. No. 11/729,505, filed March 2007 and patent application Ser. No. 11/888,059, filed Jul. 30 2007 and references cited therein, for example. In certain embodiments, the oligonucleotides may be synthesized in an array using in situ synthesis methods in which nucleotide monomers are sequentially added to a growing nucleotide chain that is attached to a solid support in the form of an array. Such in situ fabrication methods include those described in U.S. Pat. Nos. 5,449,754 and 6,180,351 as well as published PCT application no. WO 98/41531, the references cited therein, and in a variety of other publications. In one embodiment, the oligonucleotide composition may be made by fabricating an array of the oligonucleotides using in situ synthesis methods, and cleaving oligonucleotides from the array. The oligonucleotides may be amplified prior to use (e.g., by using PCR using primer sites that are at the terminal regions of the oligonucleotides, or by using polymerase promoter, e.g., a T7 polymerase promoter, that is at a terminal region of the oligonucleotides).

In certain embodiments, each of the labeled oligonucleotide probes is labeled with only one type of fluorescent moiety such that the probe only emits maximum at one wavelength. In other embodiments, each of the labeled oligonucleotide probes is labeled with two different types of fluorescent moieties such that the probe emits maxima at two different wavelengths. In certain cases, the types of fluorescent moieties used to label a set of oligonucleotide probes may be no more than six, no more than four, no more than three, or no more than two types.

Labels may be incorporated into oligonucleotide probes by any of a number of means well known to those of skill in the art. For example, the label may be simultaneously incorporated during extension or synthesis. Thus, for example, polymerase chain reaction (PCR) with labeled primers or labeled nucleotides will provide a labeled oligonucleotide product. In certain embodiments, a label may be added directly to the oligonucleotide probe. Means of attaching labels to nucleic acids are well known to those of skill in the art and include, for example nick translation or end-labeling, by kinasing of the nucleic acid and subsequent attachment of a nucleic acid linker joining the oligonucleotides to a label. Standard methods may be used for labeling the oligonucleotide, for example, as set out in Ausubel, et al, (Short Protocols in Molecular Biology, 3rd ed., Wiley & Sons, 1995) and Sambrook, et al, (Molecular Cloning: A Laboratory Manual, Third Edition, (2001) Cold Spring Harbor, N.Y.).

Detectable labels suitable for use in the present method, compositions and kits include any label detectable by spectroscopic, photochemical, biochemical, immunochemical, electrical, optical or chemical means. Useful labels include biotin for staining with labeled streptavidin conjugate, magnetic beads (e.g., DYNABEADS), fluorescent dyes (e.g., fluorescein, TEXAS RED, rhodamine, green fluorescent protein, cyanins and the like), radiolabels (e.g., 3H, 35S, 14C, or 32P, enzymes (e.g., horseradish peroxidase, alkaline phosphatase and others commonly used in ELISA), and colorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241, which are herein incorporated by reference.

In certain embodiments, the label is a fluorescent dye. Fluorescent dyes (fluorophores) suitable for use as labels in the present method can be selected from any of the many dyes suitable for use in imaging applications, especially flow cytometry. A large number of dyes are commercially available from a variety of sources, such as Molecular Probes® (Eugene, Oreg.) and Exciton (Dayton, Ohio), that provide great flexibility in selecting a set of dyes having the desired spectral properties. Examples of fluorophores include, but are not limited to, 4-acetamido-4′-isothiocyanatostilbene-2,2′ disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-amino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (TEXAS RED), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene; Alexa-Fluor dyes (e.g., Alexa Fluor 350, Alexa Fluor 430, Alexa Fluor 488, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, Alexa Fluor 750), Pacific Blue, Pacific Orange, Cascade Blue, Cascade Yellow; Quantum Dot dyes (Quantum Dot Corporation); Dylight dyes from Pierce (Rockford, Ill.), including Dylight 800, Dylight 680, Dylight 649, Dylight 633, Dylight 549, Dylight 488, Dylight 405; or combinations thereof. Other fluorophores or combinations thereof known to those skilled in the art may also be used, for example those available from Molecular Probes® (Eugene, Oreg.) and Exciton (Dayton, Ohio). Quantum dots may also be employed.

When more than one label is used, fluorescent moieties that emit different signal can be chosen such that each label can be distinctly visualized and quantified. For example, a combination of the following fluorophores may be used: 7-amino-4-methylcoumarin-3-acetic acid (AMCA), TEXAS RED (Molecular Probes, Inc.), 5-(and-6)-carboxy-X-rhodamine, lissamine rhodamine B, 5-(and-6)-carboxyfluorescein, fluorescein-5-isothiocyanate (FITC), 7-diethylaminocoumarin-3-carboxylic acid, tetramethylrhodamine-5-(and-6)-isothiocyanate, 5-(and-6)-carboxytetramethylrhodamine, 7-hydroxycoumarin-3-carboxylic acid, 6-[fluorescein 5-(and-6)-carboxamido]hexanoic acid, N-(4,4-difluoro-5,7-dimethyl-4-bora-3 a, 4a diaza-3-indacenepropionic acid, eosin-5-isothiocyanate, erythrosin-5-isothiocyanate, and CASCADE BLUE acetylazide (Molecular Probes, Inc.).

In certain embodiments, suitable distinguishable fluorescent label pairs useful in the subject methods include Cy-3 and Cy-5 (Amersham Inc., Piscataway, N.J.), Quasar 570 and Quasar 670 (Biosearch Technology, Novato Calif.), Alexa Fluor 555 and Alexa Fluor 647 (Molecular Probes, Eugene, Oreg.), BODIPY V-1002 and BODIPY V1005 (Molecular Probes, Eugene, Oreg.), POPO-3 and TOTO-3 (Molecular Probes, Eugene, Oreg.), and POPRO3 TOPRO3 (Molecular Probes, Eugene, Oreg.). Further suitable distinguishable detectable labels may be found in Kricka et al. (Ann Clin Biochem. 39:114-29, 2002). Hybridized oligonucleotides can be viewed with a fluorescence microscope and an appropriate filter for each fluorophore, or by using dual or triple band-pass filter sets to observe multiple fluorophores. See, e.g., U.S. Pat. No. 5,776,688.

Hybridized oligonucleotides also can be labeled with biotin, or digoxygenin, although secondary detection molecules or further processing may then be required to visualize the hybridized oligonucleotides and quantify the amount of hybridization. For example, an oligonucleotide labeled with biotin can be detected and quantified using avidin conjugated to a detectable enzymatic marker such as alkaline phosphatase or horseradish peroxidase. Enzymatic markers can be detected and quantified in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

Prior to in situ hybridization, the oligonucleotides may be denatured. Denaturation is typically performed by incubating in the presence of high pH, heat (e.g., temperatures from about 70° C. to about 95° C.), organic solvents such as formamide and tetraalkylammonium halides, or combinations thereof.

The oligonucleotide composition may be an aqueous composition (i.e., the oligonucleotides are dissolved in a water-based medium), or the oligonucleotides composition may be a dry composition, where the oligonucleotides may be in the form of a dry pellet.

### Kits

Also provided by the subject invention are kits for practicing the subject method, as described above. The subject kit contains a plurality of labeled oligonucleotide probes in which each of the labeled oligonucleotide probes is complementary to a non-repetitive, unique sequence in a region that flanks the centromere of a single chromosome of a plurality of chromosomes; and hybridization of the labeled oligonucleotide probes to said chromosomes produces a distinct labeling pattern for each of the chromosomes, thereby allowing each of the labeled chromosomes to be distinguished from one another; and reagents for performing fluorescent in situ hybridization.

In addition to above-mentioned components, the subject kit may further comprise information relating to labeling patterns for specific chromosomes in a plurality from a genomic sample. The information may designate an illustration or an image of a labeling pattern as a numbered chromosome such that labeled chromosomes produced by the subject method may be decoded using the provided information. In certain embodiments, the kit includes instructions for using the components of the kit to practice the subject method. The instructions for practicing the subject methods and the other relevant information are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Utility

The subject method finds use in a variety of applications, where such applications generally include genomic DNA analysis applications in which the enumeration or identification of one or more chromosomes in a given sample is desired. The subject method may also be used to identify chromosomal aberrations, such as chromosomal breakpoints, inversions, deletions and translocations in the context of a chromosome identified by its enumeration.

In general, the subject method involves the use of a set of labeled probes designed to anneal to a non-repetitive, unique sequence in a region that flanks the centromere of a single chromosome of a plurality of chromosomes such that hybridization of the labeled oligonucleotide probes to the chromosomes produces a distinct labeling pattern for each of the chromosomes, thereby allowing each of the labeled chromosomes to be distinguished from one another.

The genomic sample under study, which may or may not be suspected of containing all the correct number pairs of chromosomes expected for a supposedly wild-type organism, is contacted with the sequence-specific labeled probes. After hybridization, the labeling pattern of the hybridized probes is analyzed, as described above.

Specific analyte detection applications of interest include but are not limited to chromosomal duplications, deletions, and identifying aberrations for specific genomic loci. One embodiment of the genomic analysis assay allows the detection of a chromosomal duplication (polyploidy). In this embodiment, the assay contacts probes specific for a region of a specific chromosome under in situ hybridization conditions. If the same exact labeling pattern occurs twice in a set of plurality of chromosomes, chromosomal duplication may have occurred to a chromosome in the sample. Matching the duplicated labeling pattern to a database may provide the information necessary to enumerate and identify the chromosome that has been duplicated. Similarly, chromosomal deletion (aneuploidy) may be deciphered by enumerating all chromosomes in the plurality by matching each labeling pattern with a chromosome expected to be in the genomic sample. The labeling pattern that fails to be matched with a labeled chromosome indicates deletion of the chromosome that corresponds to the missing labeling pattern.

The subject method also finds utility in the detection of other chromosomal aberrations for one or more genomic loci. In this embodiment, the assay contacts under in situ hybridization conditions probes specific for genomic loci of interest in addition to the labeled oligonucleotide probes described above. The binding of the probes specific for genomic loci of interest may be located and analyzed in the context of a specific chromosome since all the chromosomes in the plurality can be enumerated by the subject method. For example, identifying deletion, mutation variants, amplification, or inversion of a genomic locus of interest can be facilitated by knowing the numbered chromosome to which the probes specific from the genomic locus are binding.

In addition, the subject method may lend itself to multiplexing assays since all the chromosomes can be analyzed at once and all oligonucleotide probes described herein may all be used under the same in situ hybridization conditions. In certain embodiments, there is no need to sequentially hybridize different probes for different chromosomes or to sequentially hybridize probes for genomic loci separately from probes for regions flanking the centromeres. Due to the many distinct labeling patterns that can be generated in accordance with the subject method, basic label moieties of very few types are adequate for the purposes of the assays such that complex filter sets for a detection apparatus may not be necessary. Consequently, the application of the subject method in single assays or multiplexed assays can be prevalently applied and facilitated.

The subject method finds use in a variety of diagnostic and research purposes since chromosomal aberrations play an important role in conditions relevant to human diseases and genomic evolution of many organisms.

In particular, the above-described method may be employed to diagnose, to provide a prognosis, or to investigate various types of genetic abnormalities, cancer or other mammalian diseases, including but not limited to, leukemia; breast carcinoma; prostate cancer; Alzheimer's disease; Parkinson's disease; epilepsy; amyotrophic lateral sclerosis; multiple sclerosis; stroke; autism; Cri du chat (truncation on the short arm on chromosome 5), 1p36 deletion syndrome (loss of part of the short arm of chromosome 1), Angelman syndrome (loss of part of the long arm of chromosome 15); Prader-Willi syndrome (loss of part of the short arm of chromosome 15); acute lymphoblastic leukemia and more specifically, chronic myelogenous leukemia (translocation between chromosomes 9 and 22); Velocardiofacial syndrome (loss of part of the long arm of chromosome 22); Turner syndrome (single X chromosome); Klinefelter syndrome (an extra X chromosome); Edwards syndrome (trisomy of chromosome 18); Down syndrome (trisomy of chromosome 21); Patau syndrome (trisomy of chromosome 13); and trisomies 8, 9 and 16, which generally do not survive to birth.

The disease may be genetically inherited (germline mutation) or sporadic (somatic mutation). Many exemplary chromosomal aberrations discussed herein are associated with and are thought to be a factor in producing these disorders. Knowing the type and the location of the chromosomal rearrangement may greatly aid the diagnosis, prognosis, and understanding of various mammalian diseases.

Certain of the above-described embodiments can also be used to detect diseased cells and results can be quantified automatically in a high throughput fashion by using a computer programmed to count the number and/or arrangement of labeled signals present.

The above-described methods can also be used to compare the genomes of two biological species in order to deduce evolutionary relationships.

Chromosomes may be isolated from a variety of sources, including tissue culture cells and mammalian subjects, e.g., human, primate, mouse or rat subjects. For example, chromosomes may be analyzed from less than five milliliters (mL) of peripheral blood. White blood cells contain chromosomes while red blood cells do not. Blood may be collected and combined with an anti-clotting agent such as sodium heparin. Chromosomes may also be analyzed from amniotic fluid, which contains fetal cells. Such cells can be grown in tissue culture so that dividing cells are available for chromosomal analysis within 5-10 days. Chromosomes may also be analyzed from bone marrow, which is useful for diagnosis of leukemia or other bone marrow cancers. Chromosomes may also be analyzed from solid tissue samples. A skin or other tissue biopsy in the range of about 2-3 mm may be obtained aseptically and transferred to a sterile vial containing sterile saline or tissue transport media to provide material for chromosome analysis. Fetal tissue obtained after a miscarriage can also be used for chromosome analysis, such as from the fetal side of the placenta, the periosteum overlying the sternum or fascia above the inguinal ligament, or from chorionic villi. Fetal tissue can also be collected from multiple sites such as the kidneys, thymus, lungs, diaphragm, muscles, tendons, and gonads. An amniocentesis may also be performed.

In addition to the above, the subject method may also be performed on bone marrow smears, blood smears, paraffin embedded tissue preparations, enzymatically dissociated tissue samples, uncultured bone marrow, uncultured amniocytes and cytospin preparations, for example.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of sample analysis, comprising:
a) contacting a genomic sample comprising a plurality of intact chromosomes with a first set of labeled oligonucleotide probes under in situ hybridization conditions to produce a contacted sample comprising labeled chromosomes, wherein:
i. each of said labeled oligonucleotide probes is complementary to a non-repetitive, unique sequence in a region that flanks the centromere of a single chromosome of said plurality of chromosomes; and
ii. hybridization of said labeled oligonucleotide probes to said chromosomes produces a distinct labeling pattern for each hybridized chromosome, thereby allowing each of said labeled chromosomes to be distinguished from one another;
b) imaging said hybridized chromosomes to provide an image showing the labeling pattern for each labeled chromosome; and
c) enumerating a labeled chromosome based on the labeling pattern of said labeled chromosome.

2. The method of claim 1, wherein said labeled oligonucleotide probes comprise overlapping sequences that tile across said region.

3. The method of claim 1, wherein, for at least one of said labeled chromosomes, the labeled oligonucleotide probes bind to a region that is sufficiently proximal to the centromere of said chromosome that the signals from the sister chromatids of said labeled chromosome are spatially indistinct.

4. The method of claim 1, wherein, for at least one of said labeled chromosomes, the labeled oligonucleotide probes bind to a region that is sufficiently distal to the centromere of said chromosome that the signals from the sister chromatids of said labeled chromosome are spatially distinct.

5. The method of claim 1, wherein said enumerating comprises:
a) determining whether the labeling pattern of a chromosome comprises a spatially distinct or spatially indistinct signal; and
b) determining the emission spectrum of said labeling pattern;
wherein said enumerating is based on whether said labeling pattern comprises a spatially distinct or spatially indistinct signal and the emission spectrum of said labeling pattern.

6. The method of claim 1, wherein each of said labeled oligonucleotide probes is linked to a single fluorescent moiety.

7. The method of claim 6, wherein each of said labeled chromosomes comprises a region labeled with a single fluorescent moiety or with two distinguishable fluorescent moieties.

8. The method of claim 7, wherein hybridization of said labeled oligonucleotide probes to said chromosomes produces at least 21 distinct labeling patterns for non-acrocentric chromosomes and 18 distinct labeling patterns for acrocentric chromosomes.

9. The method of claim 1, wherein said labeled oligonucleotide probes are from about 50 to about 200 nucleotides in length.

10. The method of claim 1, wherein said genomic sample is contacted with all of said labeled oligonucleotide probes at the same time.

11. The method of claim 1, wherein said contacting further comprises contacting said genomic sample with a second set of labeled probes, wherein said labeled probes of said second set are specific for genomic loci that are distal to the centromeres of said chromosomes.

12. The method of claim 11, wherein said genomic sample is contacted with said first set of labeled probes under the same in situ hybridization conditions as the in situ hybridization conditions for hybridization of said second set of labeled probes.

13. The method of claim 1, wherein said intact chromosomes are metaphase chromosomes.

14. The method of claim 1, wherein said intact chromosomes are interphase chromosomes and said method includes determining whether there are two or four regions labeled with the same label.

15. A composition comprising:
a plurality of labeled oligonucleotide probes wherein each of said labeled oligonucleotide probes is
i) complementary to a non-repetitive, unique sequence in a region that flanks the centromere of a single chromosome of a plurality of chromosomes;
ii) and hybridization of said labeled oligonucleotide probes to said chromosomes produces a distinct labeling pattern for each of said chromosomes, thereby allowing each of said labeled chromosomes to be distinguished from one another.

16. The composition of claim 15, wherein said labeled oligonucleotide probes are immobilized on a substrate.

17. The composition of claim 15, wherein said labeled oligonucleotide probes are in solution.

18. A kit for analyzing a genomic sample according to claim 1 comprising:
a) a plurality of labeled oligonucleotide probes wherein each of said labeled oligonucleotide probes is complementary to a non-repetitive, unique sequence in a region that flanks the centromere of a single chromosome of a plurality of chromosomes; and hybridization of said labeled oligonucleotide probes to said chromosomes produces a distinct labeling pattern for each of said chromosomes, thereby allowing each of said labeled chromosomes to be distinguished from one another; and
b) reagents for performing fluorescent in situ hybridization.

19. The kit of claim 18, wherein said reagents comprise a hybridization buffer

20. The kit of claim 18, wherein said reagents comprise a wash buffer.
